(19) United States
(12) Patent Application Publication
(10) Pub. No.: US 2026/0315672 A1
(43) Pub. Date: Oct. 15, 2026

(54) METHOD FOR DISINFECTING A MEDICAL DEVICE, AND MEDICAL DEVICE

(71) Applicant: B. Braun Avitum AG, Melsungen (DE)

(72) Inventors: Andre Wagner, Kassel (DE); Manuel Schaumburg, Kassel (DE); Nicolas David Mueglich, Fuldatal (DE)

(21) Appl. No.: 19/111,068

(22) PCT Filed: Sep. 13, 2023
(86) PCT No.: PCT/EP2023/075165

§ 371 Date: Mar. 12, 2025

(30) Foreign Application Priority Data
Sep. 13, 2022 (DE) 10 2022 123 274.3

(51) Int. Cl. A61M 1/16; A61L 2/18; A61L 2/26; A61L 103/15

(52) U.S. Cl. CPC A61M 1/1686; A61L 2/18; A61L 2/26; A61L 2103/15; A61L 2202/14

(57) ABSTRACT

A method is used for disinfecting a medical device, in which a temperature- and time-dependent first lethality of germs potentially present in a temperature measuring section is calculated based on a temperature measured in the temperature measuring section. A temperature- and time-dependent second lethality of germs potentially present in a predetermined volume is calculated based on a temperature allocated to the predetermined volume. A disinfection is terminated when the second lethality reaches or exceeds a second threshold value and/or an average value, calculated by the control device of the first and the second lethality reaches or exceeds a predetermined third threshold value set by the control device. A medical device can include a control device that carries out the method.

### CROSS-REFERENCE TO RELATED APPLICATIONS

This application is the United States national stage entry of International Application No. PCT/EP2023/075165, filed on Sep. 13, 2023, and claims priority to German Application No. 10 2022 123 274.3, filed on Sep. 13, 2022. The contents of International Application No. PCT/EP2023/075165 and German Application No. 10 2022 123 274.3 are incorporated by reference herein in their entireties.

### FIELD

The present disclosure relates to a method for disinfecting a medical device, preferably a dialysis machine, in which a disinfecting fluid is heated, introduced in a fluid line system of the medical device and channeled out of the fluid line system, wherein at least one temperature is measured in at least one temperature measuring section within the fluid line system by way of which a temperature- and time-dependent lethality of germs potentially present in the at least one temperature measuring section is calculated, and it is determined when the lethality reaches or exceeds a threshold value. The disclosure further relates to a medical device that is adapted to be able to carry out the method according to the disclosure.

### BACKGROUND

A generic method is disclosed, for example, in WO 2022/008 889 A1. A dialysis machine according to this document has a main body, a water purification system separate from the main body, and a fluid disinfection device arranged in the main body. A heating element and a temperature sensor are provided in the fluid disinfection device. The dialysis machine is disinfected by heating a volume of fluid by the heating element until a threshold value is exceeded, and the volume of heated fluid is conducted through a first fluid circuit comprising the main body and the fluid disinfection device, and through a second fluid circuit comprising the water purification system. During disinfection, the temperature of the heated fluid is monitored through a temperature sensor and is kept above the threshold value by means of the heating element. In order to be able to determine whether the disinfection was completed, lethality values for germs potentially present in the fluid circuits are calculated for the first and second fluid circuits, based on the measured temperature.

The problem of the conventional method of disinfection is that the calculation of lethality values is inaccurate, so that the duration for disinfection has to be set longer than necessary in order to fulfill a required minimum safety.

### SUMMARY

The purpose of the present disclosure is therefore to provide a method and a medical device, respectively, that allows for time-efficient execution of disinfection of the medical device.

A method for disinfecting a medical device according to the disclosure is particularly suited for disinfecting a blood treatment device, preferably a dialysis machine, and comprises the following steps:
heating a disinfecting fluid,
introducing the disinfecting fluid into a fluid line system of the medical device,
channeling the disinfecting fluid out of the fluid line system,
measuring at least one temperature in at least one temperature measuring section within the fluid line system,
calculating a temperature- and time-dependent first lethality of germs potentially present in the at least one temperature measuring section on the basis of the temperature measured in the at least one temperature measuring section,
determining a first threshold value for the first lethality, and
determining when the first lethality reaches or exceeds the first threshold value.

The disinfecting fluid may in particular be water with added citric or hydroxyacetic acid.

In particular, the fluid line system may comprise at least parts of a dialysis fluid circuit, a blood circuit and/or a water purification circuit or consist of at least one of these circuits.

The method according to the disclosure moreover comprises the following steps:
calculating a position-dependent temperature profile for the fluid line system, by means of which a calculated temperature can be allocated at least one predetermined volume within the fluid line system,
calculating at least one temperature- and time-dependent second lethality of germs potentially present in the at least one predetermined volume on the basis of the temperature allocated with the at least one predetermined volume,
defining a second threshold value for the at least one second lethality,
determining when the at least one second lethality reaches or exceeds the second threshold value, and
terminating disinfection when the at least one second lethality reaches or exceeds the second threshold value and/or an average value of the first and the at least one second lethality calculated by the control device (8) reaches or exceeds a third threshold value set by the control device (8).

The calculation of the position-dependent temperature profile can be carried out in particular by extrapolation based on the measured temperature. In particular, temperatures can be measured at several points in the fluid line system and a temperature profile for the fluid system can be calculated based on those several measured temperatures by means of interpolation and/or extrapolation. In particular, the temperature profile can be position- and time-dependent.

The second lethality can be calculated in particular on the basis of the same calculation type and characteristic value, respectively, as for calculating the first lethality.

The temperature can be measured especially at an inlet, an outlet, a heating element and/or a heat exchanger of the fluid line system.

The threshold for the first lethality may in particular differ from the threshold for the second lethality. Preferably, the threshold for the first lethality may be higher than the threshold for the second lethality, so that higher requirements are imposed on the first lethality compared to the second lethality. The third threshold can be determined in particular with regard to preferably mobile germs.

The threshold values can be determined in particular depending on a history of the device. If the device was used for example, for treatment of a person with chronic hepatitis, higher threshold values can be set for the (first and/or second and/or averaged) lethality.

If a temperature profile is used for disinfection, it is advantageously possible to calculate more precise values for lethality at points in the fluid line system where measuring of the temperature is not possible or only possible under aggravating conditions.

According to one aspect of the disclosure, at least one predetermined volume is or can be determined, which corresponds to a section that is stationary with respect to the fluid line system and has no temperature sensors.

If the at least one predetermined volume is stationary relative to the fluid line system, it is advantageous to monitor disinfection with respect to locally growing microorganisms at locations where no temperature sensor is positioned.

According to one aspect of the disclosure, at least one predetermined volume can be defined which is mobile with respect to the fluid line system.

If at least one predetermined volume is provided that is mobile with respect to the fluid line system, disinfection with regard to microorganisms that move or circulate in the fluid line system can be monitored in an advantageous manner.

According to one aspect of the disclosure, time-dependent changes in the temperature measured in the temperature measuring section can be considered when calculating the first lethality. If temperatures are measured at several locations (of one temperature measuring section or plural temperature measuring sections), time-dependent changes in the plural measured temperatures can be taken account of when calculating the first lethality. In other words, when calculating the first lethality for a specific time interval, temperature changes within the specific time interval can be considered.

According to one aspect of the disclosure, time-dependent changes in the temperature measured in the temperature measuring section can be taken into account when calculating the position-dependent temperature profile, so that the temperature profile on which the calculation of the at least one second lethality is based is position- and time-dependent. If temperatures are measured at several points (of a temperature measuring section or plural temperature measuring sections), time-dependent changes in the plural measured temperatures can be taken account of when calculating the position-dependent temperature profile. In other words, when calculating the position-dependent temperature profile for a specific time interval, temperature changes within the specific time interval can be taken into account.

In case changes in the measured temperature(s) when calculating the first lethality and/or the position-dependent temperature profile are considered, a time resolution of the disinfection monitoring can be improved.

According to one aspect of the disclosure, the A0 value according to ISO 15883 can be used for calculating the (first and/or second and/or averaged) lethality.

If the A0 value according to ISO 15883 is used, it is advantageous to fall back on knowledge gained from the disinfection of surgical instruments when monitoring disinfection of the fluid line system.

According to one aspect of the disclosure, a linear course can be specified for the temperature profile, at least in sections. Preferably, the entire temperature profile can have a linear course.

If a linear profile is predetermined, monitoring of disinfection can be simplified.

A medical device according to the disclosure is particularly suitable for the treatment of blood and is preferably configured as a dialysis machine. The medical device has a fluid line system, a disinfection device and a control device. The disinfection device is configured to heat a disinfecting fluid for disinfecting the fluid line system, to introduce the disinfecting fluid into the fluid line system and to channel it out of the fluid line system. The fluid line system has at least one temperature measuring section with a temperature sensor that is adapted to be able to measure a temperature in the temperature measuring section. The control device is adapted to be able to execute a method according to the disclosure.

According to one aspect of the disclosure, the medical device may have a graphical user interface that is adapted to allow different constraints and/or configurations of the medical device to be selected, and the control device can be designed to adapt the course of the temperature profile depending on the selection of at least one constraint and/or configuration.

In particular, the user interface can be adapted such that a specific air temperature and a specific air temperature range, respectively, a specific air pressure and a specific air pressure range, respectively, and/or a specific humidity and a specific humidity range, respectively, of the environment of the medical device can be input respectively selected as a boundary condition. Alternatively or additionally, the user interface can be adapted in particular such that states of the medical device in which different components are installed on the device, can be selected as configurations of the medical device. For example, the user interface can be designed such that a configuration of a dialysis machine is selectable, in which a set for a first therapy, for example a hemodialysis, is installed on the dialysis machine, and otherwise a configuration of the dialysis machine is selectable, in which a set for a second therapy, e.g. a hemodiafiltration, is installed on the dialysis machine. For different configurations of the device and respectively for different therapies of the dialysis machine corresponding temperature profiles can be stored in a database of the device and of the control device, respectively. In particular, the user interface can be a touch-sensitive screen.

If the device is designed such that the temperature profile can be adapted to different boundary conditions and/or configurations by means of a user interface, the monitoring of the disinfection can be improved.

According to one aspect of the disclosure, the medical device may comprise at least one heating device, preferably in the disinfection device, which is controllable by the control device and the control device can be designed to be able to adapt the course of the temperature profile depending on an operation of the heating device. In other words, the control device can be designed such that it already adapts the course of the temperature profile by way of instructions to the heating device. The adaptation carried out by means of the instructions can then be examined based on the subsequently measured temperature. In particular, a database can be provided in the device and respectively the control device, in which corresponding temperature profiles are stored for different instructions to the heating device.

### BRIEF DESCRIPTION OF THE DRAWINGS

The present invention will be described in more detail below by way of preferred embodiments with reference to the accompanying drawings, wherein:

FIG. 1 is a schematic view of a medical device in the form of a dialysis machine according to a first embodiment in a state in which dialysis can be carried out;

FIG. 2 is a schematic view of the dialysis machine shown in FIG. 1 in a state, in which disinfection can be performed;

FIG. 3 is a schematic detailed view of a line section marked in FIG. 2;

FIG. 4 shows the diagram of a temperature profile along the line section shown in FIG. 3;

FIG. 5 is a schematic view of the medical device in the form of a dialysis machine according to a second embodiment in a state in which dialysis can be performed;

FIG. 6 is a schematic view of the dialysis machine shown in FIG. 5 in a state in which disinfection of a dialyzing fluid circuit can be carried out;

FIG. 7 is a schematic view of the dialysis machine shown in FIG. 5 in a state in which disinfection of the blood circuit can be carried out;

FIG. 8 is a view corresponding to FIG. 6 with a marked line section of the dialyzing fluid circuit;

FIG. 9 is a view corresponding to FIG. 7 with marked line sections in the dialyzing fluid circuit and the blood circuit; and

FIG. 10 shows a diagram with a temperature profile along the line section marked in FIG. 8 and with a temperature profile along the line sections marked in FIG. 9 in the dialysis machine according to the second embodiment.

### DETAILED DESCRIPTION

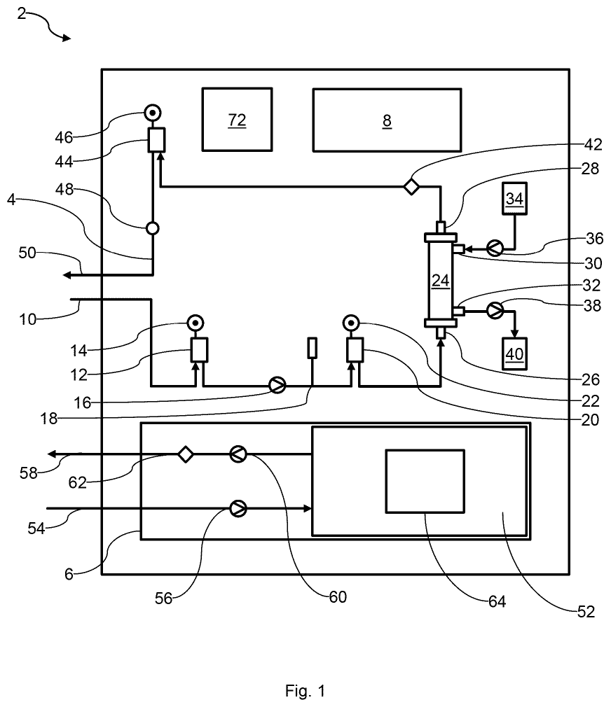

FIG. 1 shows a medical device 2 in the form of a dialysis machine, which has a fluid line system 4 in the form of a blood circuit, a disinfection device 6 and a control device 8.

The fluid line system 4 and the blood circuit, respectively, has a fluid inlet 10, into which fluid or blood can be introduced during a therapy.

Downstream of the fluid inlet 10, a first bubble chamber 12 is arranged in the fluid line system 4, which is provided with a first pressure sensor 14 in order to be able to measure the pressure of a fluid located in the first bubble chamber 12.

A fluid pump 16 is arranged in the fluid line system 4 downstream of the first bubble chamber 12 to be able to convey fluid in the fluid line system 4.

A fluid supply line 18 is arranged in the fluid line system 4 downstream of the fluid pump 16. During a therapy, an anticoagulant such as heparin can be added to the conveyed fluid or blood via the fluid supply line 18.

A second bubble chamber 20 is arranged in the fluid line system 4 downstream of the fluid supply line 18, which bubble chamber is provided with a second pressure sensor 22 in order to be able to measure the pressure of a fluid located in the second bubble chamber 20.

A fluid filter 24 is arranged in the fluid line system 4 downstream of the second bubble chamber 20. The fluid filter 24 has a fluid inlet 26, a fluid outlet 28, a dialyzing fluid inlet 30 and a dialysate outlet 32. The fluid inlet 26 and the fluid outlet 28 are connected to a fluid chamber (not shown). The dialyzing fluid inlet 30 and the dialysate outlet 32 are connected to a dialyzing fluid chamber (not shown). The fluid chamber and the dialyzing fluid chamber are in communication with each other via a semipermeable membrane (not shown). During a therapy, components such as toxins can be removed from the fluid or blood via the semipermeable membrane by diffusion and/or convection. The removed components find their way into a dialyzing fluid, which is conducted from a dialyzing fluid container 34 by means of a dialyzing fluid pump 36 into the dialyzing fluid chamber via the dialyzing fluid inlet 30, is mixed in the dialyzing fluid chamber with the components removed from the fluid or blood and is conducted together with the components removed from the fluid and blood, respectively, as dialysate from the dialysate outlet 32 into the dialysate container 40 by means of a dialysate pump 38.

Downstream of the fluid outlet 28 of the fluid filter 24, a temperature sensor 42 is arranged in the fluid line system 4, by means of which a temperature of the fluid conveyed in the fluid line system 4 can be measured.

Downstream of the temperature sensor 42, a third bubble chamber 44 is arranged in the fluid line system 4, which is provided with a third pressure sensor 46 in order to be able to measure a pressure of a fluid located in the third bubble chamber 44.

Downstream of the third bubble chamber 44, an air detector 48 is arranged in the fluid line system 4, by means of which air in the fluid conveyed in the fluid line system 4 can be detected.

Downstream of the air detector 48 a fluid outlet 50 of the fluid line system 4 is arranged, from which fluid and treated blood, respectively, can be channeled out of the fluid line system 4 during a therapy.

The disinfection system 6 has a tank 52 that is connected to an inlet line 54, via which a disinfecting fluid can be introduced into the tank 52 by means of an inlet pump 56 arranged in or on the inlet line 54. Moreover, the tank 52 is connected to an outlet line 58, via which a disinfecting fluid can be channeled out of the tank 52 by means of an outlet pump 60 arranged in or on the outlet line 58. Furthermore, downstream of the outlet pump 60, a temperature sensor 62 is arranged in or on the outlet line 58. A heating device 64 is provided in or on the tank 52, by means of which disinfecting fluid in the tank 52 can be heated or warmed.

During a therapy, the inlet line 54 and the outlet line 58 of the disinfection device 6 are not connected to the fluid line system 4.

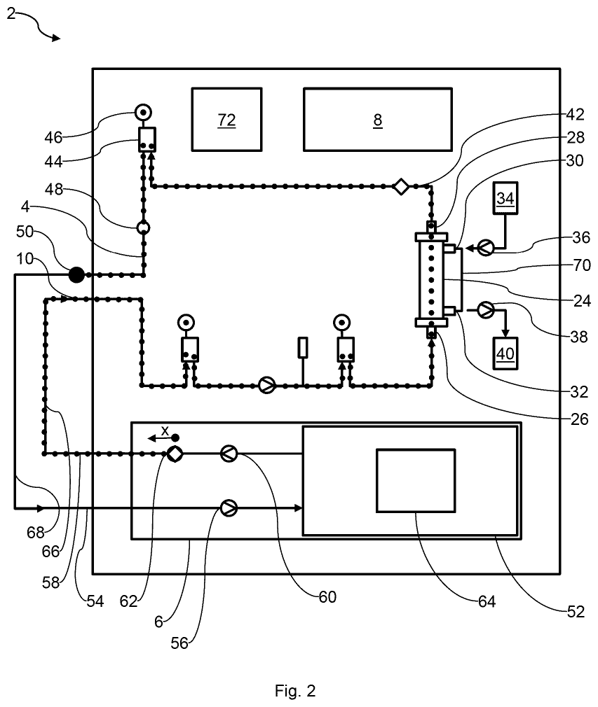

FIG. 2 shows the device according to the disclosure in a state in which the fluid line system 4 can be disinfected by means of the disinfection device 6.

In order to be able to disinfect the fluid line system 4 prior to a therapy, the outlet line 58 of the disinfection device 6 is connected to the fluid inlet 10 of the fluid line system 4 by means of a first rinsing bridge 66, the inlet line 54 of the disinfection device 6 is connected to the fluid outlet 50 of the fluid line system 4 by means of a second rinsing bridge 68, and the dialyzing fluid inlet 30 of the fluid filter 24 is connected to the dialysate outlet 32 of the fluid filter 24 by way of a third rinsing bridge 70.

The rinsing bridges 66, 68 and 70 can be implemented as separate tubes or lines and line sections, respectively, that can be manually attached to the corresponding connections of the fluid line system 4 and the respective disinfection device 6. Alternatively or additionally, the device may have multi-port valves 4 at connections 10, 30, 32 and 50 of the fluid line system 4.

The multi-port valves at connections 10 and 50 can be configured such that, in a therapy position, on the one hand, they connect the fluid inlet 10 of the fluid line system 4 to a blood outlet of a person to be treated and the fluid outlet 50 of the fluid line system 4 to a blood inlet of the person to be treated, and, on the other hand, in a disinfection position, they connect the fluid inlet 10 of the fluid line system 4 to the outlet line 58 of the disinfection device 6, and the fluid outlet 50 of the fluid line system 4 to the inlet line 54 of the disinfection device 6.

The multi-port valves at connections 30 and 32 can be designed such that, on the one hand, in a therapy position, they connect the dialyzing fluid inlet 30 of the fluid filter 24 to the dialyzing fluid container 24, and the dialysate outlet 32 of the fluid filter 24 to the dialysate container 40 and, on the other hand, in a disinfection position, they connect the dialyzing fluid inlet 30 of the fluid filter 24 to the dialysate outlet 32 of the fluid filter 24.

If multi-port valves are provided, switching of the device from a therapy state, in which therapy in the form of dialysis can be carried out, to a disinfection state, in which disinfection can be carried out, can be designed in such a way that no manual retooling is necessary.

During a disinfection, disinfecting fluid in the tank 52 is heated by means of the heating device 64 and conveyed by way of the outlet pump 60 into the fluid line system 4 via the rinsing bridge 66. In the fluid line system 4, the heated disinfecting fluid flows through the first bubble chamber 12 before being conveyed to the fluid filter 24 by the fluid pump 16 via the second bubble chamber 20. The rinsing bridge 70 ensures that the disinfecting fluid only leaves the fluid filter 24 via the fluid outlet 28. From the fluid outlet 28, the disinfecting fluid flows over the temperature sensor 42, the third bubble chamber 44 and the rinsing bridge 68 back into the tank 52, with the inlet pump 56 pumping the disinfecting fluid into the tank 52. The heat introduced into the fluid line system 4 by the disinfecting fluid kills any germs potentially present in the fluid line system 4.

In order to evaluate a disinfection of the device 2 according to the disclosure, the lethality of germs potentially present in a relevant line section of the device 2 is calculated using the A0-value concept according to ISO 15883.

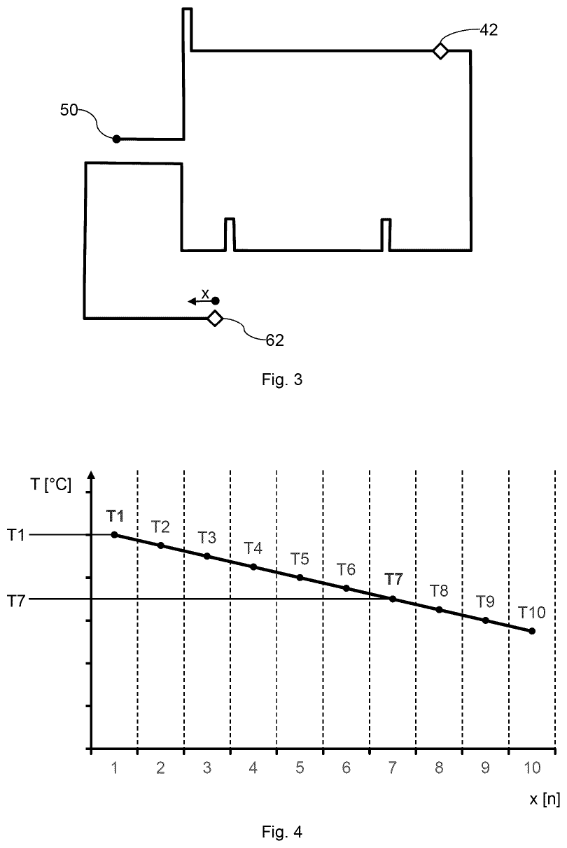

FIG. 3 shows a schematic detailed view of the line section marked with a dotted line in FIG. 2, which extends, in the disinfection state of the device 2, from the temperature sensor 62 of the disinfection device 6 to the fluid outlet 50 of the fluid line system 4.

The A0 value is a measure of lethality and is calculated according to ISO 15883 using the following formula:

$${A}_{0} = \sum 1 {0}^{\frac{[ T ( t ) - 80 °C . ]}{z}} \cdot Δ t$$

“z” is the z-value, for the definition of which ISO 11139, for example, can be fallen back to. For the A0 value, “z=10° C.” applies.
“T(t)” is the temperature of the disinfecting fluid in ° C. as a function of time “t”.
“Δt” is the selected time interval in seconds.

In order to enable time-efficient disinfection, not the A0 value of the entire line section is calculated generally using only the two temperatures measured by temperature sensors 42 and 62. Rather, according to the disclosure, the line section and fluid line system 4 and the relevant part of the fluid line system 4, respectively, is first divided into k subsections. For example, the line section as shown in FIG. 4 is divided into k=10 subsections.

Those subsections of the line section or fluid line system 4 that extend at temperature sensors 42 and 62 are, according to the disclosure, temperature measuring sections. To calculate the A0 values of the temperature measuring sections, temperature T7 measured by temperature sensor 42 and temperature T1 measured by temperature sensor 62 can be used directly. The A0 values of the temperature measuring sections each correspond to a first lethality according to the disclosure.

In order to be able to also calculate A0 values in the line section away from temperature sensors 62 and 42, a temperature profile is calculated based on measured temperatures T1 and T7. For example, as shown in FIG. 4, a linear course of the temperature is assumed. Depending on the position x in the line section and the time t, the temperature can be calculated using the following formula:

$$T ( x , t ) = {T}_{1} ( t ) + \frac{{T}_{7} ( t ) - {T}_{1} ( t )}{{x}_{7} - {x}_{1}} \cdot ( x - {x}_{1} )$$

On the basis of temperatures T2 to T6 and T8 to T10 calculated by this formula, corresponding A0 values can be calculated for the sections away from the temperature measuring sections. These A0 values off the temperature measuring sections specified by temperature sensors 42 and 62 in each case correspond to a second lethality according to the disclosure. The volumes in the sections remote from the temperature measuring sections correspond to predetermined volumes according to the disclosure.

To obtain an A0 value for the entire line section shown in FIG. 3, an averaged A0 value can be calculated from the calculated temperature profile using the following formula:

$${\overline{A}}_{0} = {\sum }_{0}^{t} \frac{1}{k} {\sum }_{n = 1}^{k} 1 {0}^{[ T ( n , t ) - 80 °C . ] / z}$$

For the example shown in FIG. 4, “k=10” applies.

The A0 values for the temperature measuring sections, the portions of the line section shown in FIG. 3 that are off the temperature measuring sections, and the averaged A0 value are calculated by the control device 8, which is connected to the actuators (fluid pump 16, dialyzing fluid pump 36, dialysate pump 38, inlet pump 56, outlet pump 60 and heating device 64), and sensors (pressure sensors 14, 22 and 46, temperature sensors 42 and 62, and air detector 48) of the device 2 via signals.

In order to be able to define threshold values for the A0 values, the device 2 has a touch-sensitive screen 72 that is connected to the control device 8 by signals.

At least one threshold value for the A0 values of the temperature measuring sections, at least one threshold value for the subsections off the temperature measuring sections and at least one threshold value for the averaged A0 value of the line section can be input onto the screen 72. Depending on the input on screen 72, the control device 8 determines the corresponding threshold values.

During disinfection, the control unit 8 continuously compares the calculated A0 values for the temperature measuring sections and for the subsections off the temperature measuring sections, as well as the calculated averaged A0 value, with the corresponding previously defined threshold values. Once at any point in time the calculated A0 values for the temperature measuring sections and for the subsections off the temperature measuring sections, as well as the calculated averaged A0 value reach or exceed the corresponding threshold values, the control unit 8 terminates the disinfection by stopping the pumps 60, 16 and 56.

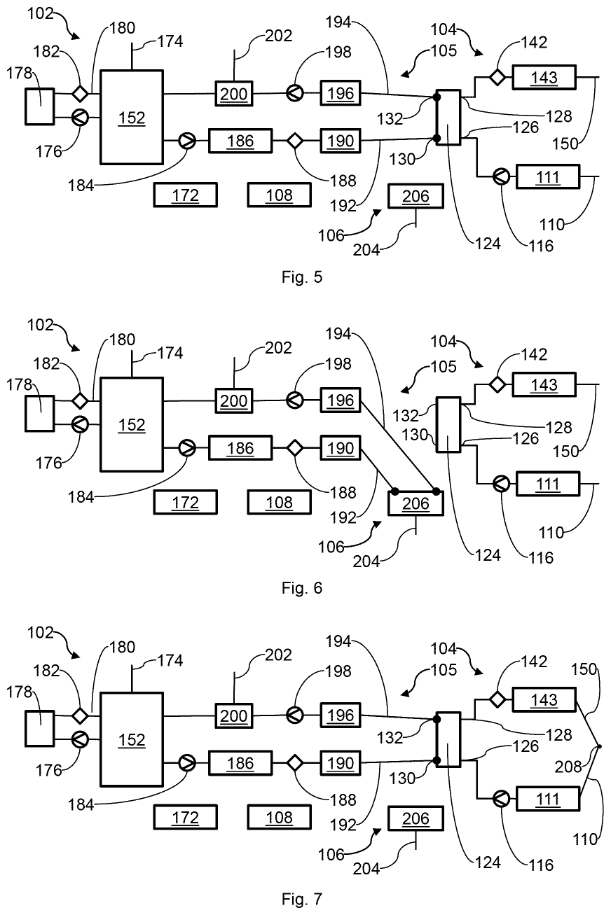

FIG. 5 shows a medical device 102 in the form of a dialysis machine, which has a fluid line system 104 in the form of a blood circuit, a fluid line system 105 in the form of a dialyzing fluid circuit, a disinfection device 106 and a control device 108, as used during a therapy.

The fluid line system 104 and the blood circuit, respectively, can be a part of the dialysis machine or a reusable and separately mountable set of blood tubing and dialyzer.

The fluid line system 104 and the blood circuit, respectively, has a fluid inlet 110 into which fluid or, during a therapy, blood can be introduced.

Downstream of fluid inlet 110, various functional components are arranged in the fluid line system 104, which are not of primary importance for the present disclosure. These are summarized as components 111.

Downstream of 111, a pump 116 is arranged, which pumps the fluid in the fluid line system 104.

A fluid filter 124 is arranged in the fluid line system 104 downstream of the pump 116. The fluid filter 124 has four fluid connections. Two are for the inlet (126) and outlet (128) of the blood circuit and two are for the inlet (130) and outlet (132) of the dialyzing fluid circuit. The circuits for blood and dialyzing fluid are in communication with each other via a semipermeable membrane (not shown). During a therapy, components such as toxins can be extracted from the fluid or blood via the semipermeable membrane by diffusion and/or convection. During this process, the removed components find their way from the blood circuit into the dialyzing fluid.

Downstream of the fluid outlet 128 of fluid filter 124, a temperature sensor 142 is arranged in the fluid line system 104, by means of which a temperature of the fluid conveyed in the fluid line system 104 can be measured.

Downstream of the temperature sensor 142, various functional components are arranged in the fluid line system 104, which are not of primary importance for the present disclosure. These are summarized as components 143.

Downstream of the components 143 a fluid outlet 150 of the fluid line system 104 is arranged, from which fluid or blood treated during a therapy can be discharged from the fluid line system 104.

The fluid line system 105 or the dialyzing fluid circuit has a fluid inlet 174 into which water can be introduced.

Downstream of the fluid inlet 174 is a fluid tank 152 in which fluid or, during therapy, dialyzing fluid is collected.

A pump 176 can take fluid from the fluid tank 152 and convey it to a heater 178 in a heating circuit. The heater 178 is used to heat the fluid, which is then pumped back to the fluid tank 152 via a line 180.

A temperature sensor 182 is included in the line 180, which can be configured as tube element W6, for example, which determines the temperature of the fluid in the dialyzing fluid circuit 105 after initial heating.

A pump 184 can take fluid out of the fluid tank 152 and pump it in the direction of the blood circuit 104. Downstream of the pump 184, various functional components of the dialyzing fluid circuit are arranged, which, with the exception of temperature sensor 188, are not of primary importance for the present disclosure. These components are summarized by means of 186 and 190. The temperature sensor 188 is used to determine the temperature of the fluid that is pumped towards the blood circuit 104.

Downstream of components 190, the fluid is conducted via a line 192, which may be designed as a tube, to the fluid filter 124. The line 192 can be connected to the fluid filter 124 via a coupling on the fluid connection 130.

Downstream of the fluid filter 124, the fluid can be directed away from the fluid filter 124 via a line 196, which can be designed as a tube. The line 194 can be connected to the fluid filter 124 via a coupling.

Downstream of the line 194, the fluid is directed over various components which are not of primary importance for the present disclosure. These components are summarized as 196.

Downstream of the components 196, a pump 198 is arranged which pumps the fluid back into the fluid tank 152. The fluid tank 152 is designed so that the discharged fluid does not have to come into contact with the incoming liquid.

A valve 200 is arranged downstream of the pump 198. In the therapy mode, the valve 200 directs the fluid towards an outlet 202.

The disinfection device 106 has an inlet or intake 204 through which disinfectant can be introduced. The disinfection device 106 is not necessarily connected during therapy.

FIG. 6 shows the medical device 102 during disinfection of the dialyzing fluid circuit 105.

For disinfection, different phases have to be distinguished.

In the first phase of disinfection, the disinfectant is introduced.

For disinfection, a disinfectant is introduced via the inlet 204. For this, the lines 192 and 194 are connected to the disinfection device 106 with their couplings. A three-way valve 206 of the disinfection device 106 can be operated in different configurations. During the introduction of the disinfectant, it connects the inlet 204 to the line 194. The pump 198 can thus introduce the disinfectant into the circuit 105. Downstream of the pump 198, the valve 200 is set so as to direct the disinfectant into the fluid tank 152. There it is mixed with water from the fluid inlet 174. In the following, this mixture is referred to as disinfecting fluid.

In the second phase of the disinfection, the disinfecting fluid is heated. For this purpose, the fluid is pumped by the pump 176 from the fluid tank 152 via a heating element of the heater 178. The temperature of the disinfecting fluid is continuously determined through temperature sensor 182.

The fluid can be circulated continuously and repeatedly through the heating element of the heater 178 to reach the required temperature.

In the third phase of the disinfection, the heated disinfecting fluid is circulated in the dialyzing fluid circuit 105. This is where the actual disinfection of the dialyzing fluid circuit 105 takes place.

For this, the same actuators as in the therapy phase can be used to distribute the fluid in the dialyzing fluid circuit 105. The pump 184 conveys the disinfecting fluid from the fluid tank 152 via the component(s) 186, the temperature sensor 188 and the component(s) 190. The temperature sensor 188 allows a measurement of the temperature of the disinfecting fluid. This measured value is used to extrapolate the temperature in other areas of the dialyzing fluid circuit 105.

Downstream of component(s) 190, the fluid is directed to the disinfection device 106. During this phase, the three-way valve 206 is set so that the fluid continues to be passed on to the line 194. Via the component(s) 196, the pump 198 and the valve 200, the fluid is returned to the fluid tank 152.

The temperature of the disinfecting fluid decreases continuously during the flow path, so that the disinfecting effect is increasingly reduced.

The heating circuit with elements 176 to 182 continues to remain active during this phase in order to maintain the temperature in the fluid tank 152. In this phase, the disinfecting fluid can be circulated through the fluid system until the desired lethality values are obtained at each location.

FIG. 7 shows the medical device 102 during disinfection of the blood circuit 104.

In the (optional) fourth phase of disinfection, the blood circuit 104 can be disinfected in addition to the dialyzing fluid side. The circulation of the disinfecting fluid from phase 3 of the disinfection continues. In this phase, however, the couplings of lines 192 and 194 are connected again to the fluid filter 124.

This flushes the fluid filter 124 with the heated disinfecting fluid. The disinfectant can also penetrate the blood circuit 104 via the semipermeable membrane.

In this phase, the fluid inlet 110 and the fluid outlet 150 of the blood circuit 104 are connected to one another by means of a coupling 208.

The pump 116 is used in this phase to circulate the fluid in the blood circuit 104. The temperature in the blood circuit 104 is continuously increased by the substance and heat exchange in the fluid filter 124. The temperature sensor 142 measures the temperature in the blood circuit 104 and can be used to extrapolate the temperature in other areas of the blood circuit 104.

In order to be able to evaluate a disinfection of the device 102 according to the disclosure, the lethality of germs potentially present in a relevant line section of the device 102 is calculated using the A0-value concept according to ISO 15883.

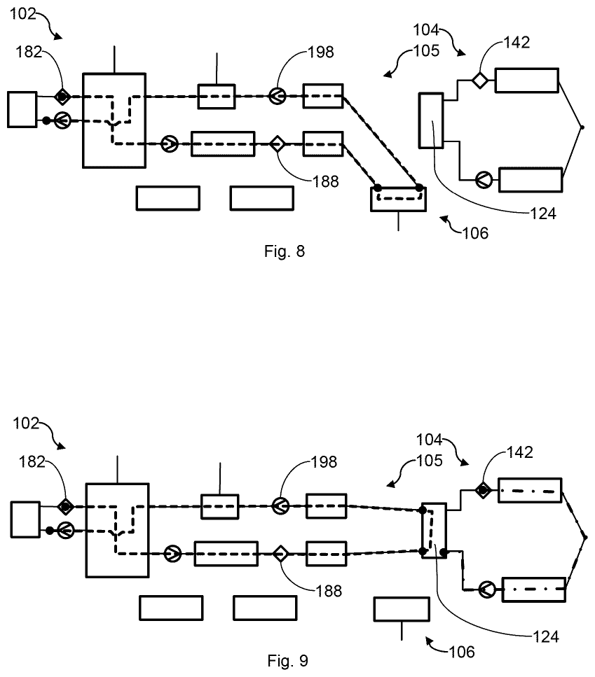

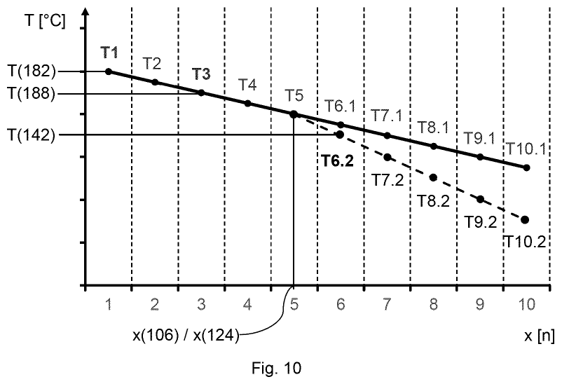

FIG. 8 shows a view corresponding to FIG. 6 with a marked line section of the dialyzing fluid circuit 105 during disinfection of the dialyzing fluid circuit 105. FIG. 9 shows a view corresponding to FIG. 7 with marked line sections in the dialyzing fluid circuit 105 and the blood circuit 104 in the case of additional disinfection of the blood circuit 104.

FIG. 10 shows a schematic detailed view of the temperature course in the line sections of the dialyzing fluid circuit 105 and the blood circuit 104 marked in FIGS. 8 and 9. The temperature course of the line section in the dialyzing fluid circuit 105 is depicted by a solid line and the temperature curve of the line section in the blood circuit 104 is shown with a dashed line. The highest temperature is reached in the heating element of the heater 178 and from there, decreases in the course of the fluid path x. At the positions of the temperature sensors 182 and 188, the temperature is known from measurements. With these measured values, the temperature in other areas of the dialyzing fluid circuit 105 can be extrapolated.

The temperature course in the blood circuit 104 results from the temperature course in the dialyzing fluid circuit. In the area of the fluid filter 124, the temperatures in the dialyzing fluid circuit 105 and in the blood circuit 104 are almost identical, since the fluid filter 124 is a very effective heat exchanger due to its semipermeable membrane. In the blood circuit 104, the temperature moreover is known for the position respectively section of the temperature sensor 142. The temperature in the remaining areas of the blood circuit 104 can be extrapolated from these two points (i.e., fluid filter 124 and temperature sensor 142).

As in the first embodiment, the A0 value is used as a measure of lethality and is calculated according to ISO 15883 by the following formula:

$${A}_{0} = \sum 1 {0}^{\frac{[ T ( t ) - 80 °C . ]}{z}} \cdot Δ t$$

“z” is the z-value, for the definition of which ISO 11139, for example, can be resorted to. For the A0 value, “z=10° C.” applies.
“T(t)” is the temperature of the disinfecting fluid in ° C. as a function of time “t”.
“Δt” is the selected time interval in seconds.

To enable time-efficient disinfection, the A0 value of the entire line section is not calculated generally merely on the basis of temperatures T1, T3 and T6.2 measured by temperature sensors 182, 188 and 142. Rather, according to the disclosure, the section of the dialyzing fluid circuit 105 to be disinfected (see FIG. 8 and the solid line in FIG. 10) is first divided into k subsections. For example, as shown in FIG. 10, the line section is divided into k=10 subsections. If the blood circuit is disinfected, the line section of the dialyzing fluid circuit 105, starting from the temperature sensor 182 up to the fluid filter 124 and then the line section of the blood circuit 104 are considered together and divided into k subsections (see FIG. 9 and in FIG. 10, the solid line to x(124) and the dashed line from x(124)).

Those subsections of the line section and fluid line systems 104 and 105 that extend at temperature sensors 142, 182 and 188, are temperature measuring sections according to the disclosure. To calculate the A0 values of the temperature measuring sections, the temperatures measured by the temperature sensors 142, 182 and 188 (e.g. T1=T(182), T3=T(188) and T6.2=T(142)) can be used directly. The A0 values of the temperature measuring sections each correspond to a first lethality according to the disclosure.

In order to be able to calculate A0 values in the line section off the temperature sensors 142, 182 and 188, a temperature profile for the dialyzing fluid circuit 105 and for the blood circuit 104 is calculated on the basis of measured temperatures T1=T(182), T3=T(188) and T6. 2=T(142). For example, as shown in FIG. 10, a linear course of the temperature is assumed. Depending on a position x in the line section and on the time t, the temperature can be calculated by the following formula:

$$T ( x , t ) = {T}_{1} ( t ) + \frac{{T}_{7} ( t ) - {T}_{1} ( t )}{{x}_{7} - {x}_{1}} \cdot ( x - {x}_{1} )$$

Based on the temperatures T2, T4, T5 and T6.1 to T10.1 and T2, T4, T5 and T6.2 to T10.2, respectively, calculated by this formula, corresponding A0 values can be calculated for the portions away from the temperature measurement sections. These A0 values off the temperature measurement sections specified by temperature sensors 142, 182 and 188 each correspond to a second lethality according to the disclosure. The volumes in the portions off the temperature measurement sections correspond to predetermined volumes according to the disclosure.

To obtain an A0 value for the entire line section shown in FIG. 8 or FIG. 9, an averaged A0 value can be calculated from the calculated temperature profile using the following formula:

$${\overline{A}}_{0} = {\sum }_{0}^{t} \frac{1}{k} {\sum }_{n = 1}^{k} 1 {0}^{[ T ( n , t ) - 80 °C . ] / z}$$

For the examples shown in FIG. 10, “k=10” applies.

The A0 values for the temperature measuring sections, the portions of the line sections shown in FIG. 8 and FIG. 9 off the temperature measuring sections, and the averaged A0 value are calculated by the control device 108, which is connected to the actuators (e.g. pumps 176, 184, 198 and 116) and the sensors (e.g. temperature sensors 142, 182 and 188) of the device 102 by means of signals.

In order to be able to set threshold values for the A0 values, the device 102 has a touch-sensitive screen 172 that is connected to the control device 108 by means of signals.

On the screen 172, at least one threshold value for the A0 values of the temperature measuring sections, at least one threshold value for the subsections away from the temperature measuring sections, and at least one threshold value for the averaged A0 value of the line section can be input. Depending on the input on screen 172, the control device 108 determines the corresponding threshold values.

During disinfection, the control device 108 continuously compares the calculated A0 values for the temperature measuring sections and for the subsections off the temperature measuring sections, as well as the calculated averaged A0 value with the corresponding previously defined threshold values. As soon as the calculated A0 values for the temperature measuring sections and for the subsections off the temperature measuring sections, as well as the calculated averaged A0 value, reach or exceed the corresponding threshold values at any point of time, the control device 108 terminates disinfection by starting to rinse out the disinfectant (e.g. by means of pumps 176, 184, 198 and 116) and switching off the heater 178.

### LIST OF REFERENCE SIGNS

2; 102 Device
4; 104, 105 Fluid line system
6; 106 Disinfection device
8; 108 Control device
10; 110 Fluid inlet of the fluid line system
12 First bubble chamber
14 First pressure sensor
16; 116 Fluid pump
18 Fluid supply line
20 Second bubble chamber
22 Second pressure sensor
24; 124 Fluid filter
26; 126 Fluid inlet of the fluid filter
28; 128 Fluid outlet of the fluid filter
30; 130 Dialyzing fluid inlet
32; 132 Dialysate outlet
34 Dialyzing fluid container
36 Dialyzing fluid pump
38 Dialysate pump
40 Dialysate container
42; 124 Temperature sensor in the fluid line system
44 Third bubble chamber
46 Third pressure sensor
48 Air detector
50; 150 Fluid outlet of the fluid line system
52; 152 Tank
54 Inlet line
56 Inlet pump
58 Outlet line
60 Outlet pump
62 Temperature sensor in the disinfection device
64 Heating system
66 First rinsing bridge
68 Second rinsing bridge
70 Third rinsing bridge
72; 172 Touch-sensitive screen
111 Component
143 Component
174 Fluid inlet
176 Pump
178 Heater
180 Line
182 Temperature sensor
184 Pump
186; 190 Component
188 Temperature sensor
192, 194 Line
196 Component
198 Pump
200 Valve
202 Outlet
204 Inlet
206 Three-way valve
208 Coupling

1.-10. (canceled)

11. A method for disinfecting a medical device comprising the steps of:
heating a disinfecting fluid;
introducing the disinfecting fluid into a fluid line system of the medical device;
channeling the disinfecting fluid out of the fluid line system;
measuring at least one temperature in at least one temperature measuring section within the fluid line system;
calculating a first lethality of germs potentially present in the at least one temperature measuring section based on the at least one temperature measured in the at least one temperature measuring section, the first lethality being temperature-dependent and time-dependent;
determining a first threshold value for the first lethality;
detecting when the first lethality reaches or exceeds the first threshold value;
calculating a position-dependent temperature profile for the fluid line system by which a calculated temperature is allocatable to at least one predetermined volume within the fluid line system;
calculating at least one second lethality of potentially present germs in the at least one predetermined volume based on the calculated temperature, the at least one second lethality being temperature-dependent and time-dependent;
defining a second threshold value for the at least one second lethality;
determining when the at least one second lethality reaches or exceeds the second threshold value; and
terminating a disinfection when the at least one second lethality reaches or exceeds the second threshold value and/or an average value calculated by a control device, of the first lethality and the at least one second lethality reaches or exceeds a third threshold value set by the control device.

12. The method according to claim 11, wherein at least one predetermined volume is defined that corresponds to a section that is stationary with respect to the fluid line system and that has no temperature sensors.

13. The method according to claim 11, wherein at least one predetermined volume is defined that is mobile with respect to the fluid line system.

14. The method according to claim 11, wherein time-dependent changes in the at least one temperature measured in the at least one temperature measuring section are accounted for when calculating the first lethality.

15. The method according to claim 11, wherein time-dependent changes in the at least one temperature measured in the at least one temperature measuring section are accounted for when calculating the position-dependent temperature profile, so that the position-dependent temperature profile on which calculation of the at least one second lethality is based, is position-dependent and time-dependent.

16. The method according to claim 11, wherein an A0 value according to ISO 15883 is used for calculating the first lethality.

17. The method according to claim 11, wherein a linear profile is specified for the position-dependent temperature profile at least in sections.

18. A medical device comprising:
a control device adapted to carry out the method according to claim 11;
a fluid line system; and
a disinfection device for disinfecting the fluid line system,
the disinfection device configured to heat a disinfecting fluid, introduce the disinfecting fluid into the fluid line system, and channel the disinfecting fluid out of the fluid line system, and
the fluid line system having at least one temperature measuring section with a temperature sensor configured to measure a temperature in the at least one temperature measuring section.

19. The medical device according to claim 18, further comprising a graphical user interface configured to allow different boundary conditions and/or configurations of the medical device to be selected, wherein the control device is configured to adjust a course of the position-dependent temperature profile depending on a selection of at least one boundary condition and/or at least one configuration.

20. The medical device according to claim 18, further comprising at least one heater controllable by the control device, wherein the control device is configured to adjust a course of the position-dependent temperature profile depending on operation of the at least one heater.
